(19) United States
(12) Patent Application Publication
(10) Pub. No.: US 2011/0255658 A1
(43) Pub. Date: Oct. 20, 2011

(54) X-RAY CT APPARATUS AND X-RAY DETECTOR

(75) Inventors: Keiji MATSUDA, Nasushiobara-Shi (JP); Yoshiaki Yaoi, Nasushiobara-Shi (JP); Machiko Iso, Nasushiobara-Shi (JP); Shuya Nambu, Nasushiobara-Shi (JP); Takashi Kanemaru, Nasushiobara-Shi (JP); Akihiko Taniguchi, Yokohama-Shi (JP)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 13/089,824

(22) Filed: Apr. 19, 2011

(30) Foreign Application Priority Data
Apr. 20, 2010 (JP) 2010-097330

(51) Int. Cl. A61B 6/03; G01T 1/20

(52) U.S. Cl. 378/19; 250/366

(57) ABSTRACT

An X-ray detector of an X-ray CT apparatus has a plurality of collimators, a plurality of scintillators, a plurality of photodiodes and a light guide. The plurality of collimators collimate an X-ray. The plurality of scintillators respectively emit fluorescence based on the X-ray from the plurality of collimators. The plurality of photodiodes respectively convert the fluorescence from the plurality of scintillators into the electric signal. The light guide has such a shape as to guide the fluorescence emitted from the plurality of scintillators respectively to the plurality of photodiodes while focusing the fluorescence toward the plurality of photodiodes.

### CROSS-REFERENCE TO RELATED APPLICATION

This application is based upon and claims the benefit of priority from Japanese Patent Application No. 2010-97330, filed on Apr. 20, 2010, the entire contents of which are incorporated herein by reference.

### FIELD

The present embodiment relates to an X-ray CT apparatus and an X-ray detector where a PDA (photodiode array) is arranged.

### BACKGROUND

X-ray CT apparatuses have an X-ray source and an X-ray detector arranged facing each other with an object therebetween. The X-ray detector includes a′plurality of channels (N channels) of detection elements along a direction (a channel direction) perpendicular to a longitudinal direction of a table-top as a body axis direction.

While various types of X-ray detectors may be employed, a scintillation detector, which can be small-sized, is typically used in the X-ray CT apparatus. Each detection element of the scintillation detector has a scintillator and an optical sensor such as a PD. The scintillator absorbs X-rays collimated at a preceding stage to thereby produce fluorescence. The PD converts the fluorescence into an electric signal by the optical sensor, and outputs the electric signal to a data acquisition system (DAS). In the X-ray CT apparatus, the X-ray source emits an X-ray beam in a fan shape toward a section of an object, and each detection element of the X-ray detector converts the X-ray beam transmitted through a slice surface of the object into the electric signal. Transmission data can be thereby collected.

As compared to the above single-slice X-ray CT apparatus, in a multi-slice X-ray CT apparatus, an X-ray detector includes not only the N channels of detection elements, but also a plurality of rows (M rows) of detection elements along the body axis direction of an object. The X-ray detector of the multi-slice X-ray CT apparatus is a two-dimensional detector for X-ray CT having the N channels×M rows of detection elements as a whole.

In a conventional X-ray CT apparatus, all PDAs (a plurality of PDs) are in contact with a working area that is not adjacent to another PDA. All the PDAs can be thereby arrayed using the working area in a process of arraying the PDAs in the X-ray CT apparatus.

However, since there is a minimum gap necessary for arraying the PDAs between respective adjacent PDAs, the PDAs are difficult to accurately array in a two-dimensional direction in the process of arraying the PDAs in the X-ray CT apparatus. Especially when a plurality of PDAs are arranged in the channel direction and three or more PDAs are arranged in a row direction, some of the PDAs (a shaded area in FIG. 3) are not in contact with the working area. The PDA not in contact with the working area is entirely surrounded by other PDAs. In the process of arraying the PDAs in the X-ray CT apparatus, the PDAs not in contact with the working area are difficult to array when there is only a minimum gap necessary for arraying the PDAs in contact with the working area. To solve the problem, the gap between the PDAs is made larger than the minimum gap necessary for arraying the PDAs in contact with the working area, so that the PDAs not in contact with the working area can be accurately arrayed. When the gap between the PDAs is made larger than the minimum gap necessary for arraying the PDAs in contact with the working area, however, detection efficiency of X-rays is reduced.

When one scintillator is provided corresponding to one PDA, fluorescence is spread inside the scintillator due to isotropic light emission within the scintillator, thereby causing a problem that an image is blurred.

The scintillator may have a convergent shape (a tapered shape). However, in this case, there is a problem that X-rays are transmitted to the PDA side since an end side of the converging type scintillator has a smaller scintillator thickness than that of a center side so as not to fully absorb X-rays.

### BRIEF DESCRIPTION OF THE DRAWINGS

In accompanying drawings,

FIG. 1 is a hardware configuration diagram illustrating an X-ray CT apparatus according to the present embodiment;

FIG. 2 is a top view (an X-ray incident surface) and a side view illustrating a first configuration of an X-ray detector of a conventional X-ray CT apparatus;

FIG. 3 is a top view (an X-ray incident surface) and a side view illustrating a second configuration of an X-ray detector of a conventional X-ray CT apparatus;

FIG. 4 is a top view (an X-ray incident surface) and a side view illustrating a configuration of an X-ray detector of the X-ray CT apparatus according to the present embodiment;

FIG. 5 is an enlarged side view illustrating a first configuration example of the X-ray detector of the X-ray CT apparatus according to the present embodiment; and

FIG. 6 is an enlarged side view illustrating a second configuration example of the X-ray detector of the X-ray CT apparatus according to the present embodiment.

### DETAILED DESCRIPTION

An X-ray CT apparatus and an X-ray detector according to the present embodiment will be described by reference to the accompanying drawings.

To solve the above-described problems, the X-ray CT apparatus according to the present embodiment has: an X-ray source configured to generate an X-ray; and an X-ray detector configured to acquire an electric signal based on the X-ray, wherein the X-ray detector has: a plurality of collimators configured to collimate the X-ray; a plurality of scintillators respectively configured to emit fluorescence based on the X-ray from the plurality of collimators; a plurality of photodiodes respectively configured to convert the fluorescence from the plurality of scintillators into the electric signal; and a light guide having such a shape as to guide the fluorescence emitted from the plurality of scintillators respectively to the plurality of photodiodes while focusing the fluorescence toward the plurality of photodiodes.

To solve the above-described problems, the X-ray detector according to the present embodiment has: a plurality of collimators configured to collimate an X-ray; a plurality of scintillators respectively configured to emit fluorescence based on the X-ray from the plurality of collimators; a plurality of photodiodes respectively configured to convert the fluorescence from the plurality of scintillators into the electric signal; and a light guide having such a shape as to guide the fluorescence emitted from the plurality of scintillators respectively to the plurality of photodiodes while focusing the fluorescence toward the plurality of photodiodes.

There are various types of the X-ray CT apparatus of the present embodiment, such as a ROTATE/ROTATE type in which an X-ray tube and an X-ray detector rotate as one body around an object, a STATIONARY/ROTATE type in which a large number of detection elements are arrayed in a ring-shape, and only the X-ray tube rotates around the object, and the like. The present invention can be applied to any of those types. Hereafter, the ROTATE/ROTATE type which is currently in a mainstream position will be described.

In addition, in recent years, a progress has been made in the commercialization of a so-called multi-tube type X-ray CT apparatus, in which a plurality of pairs of the X-ray tube and the X-ray detector are mounted on a rotary ring, and the development of peripheral technologies thereof has been in progress. The X-ray CT apparatus of the present embodiment can be applied to either of a conventional single-tube type X-ray CT apparatus, or a multi-tube type X-ray CT apparatus. Here, description will be made supposing a single-tube type X-ray CT apparatus.

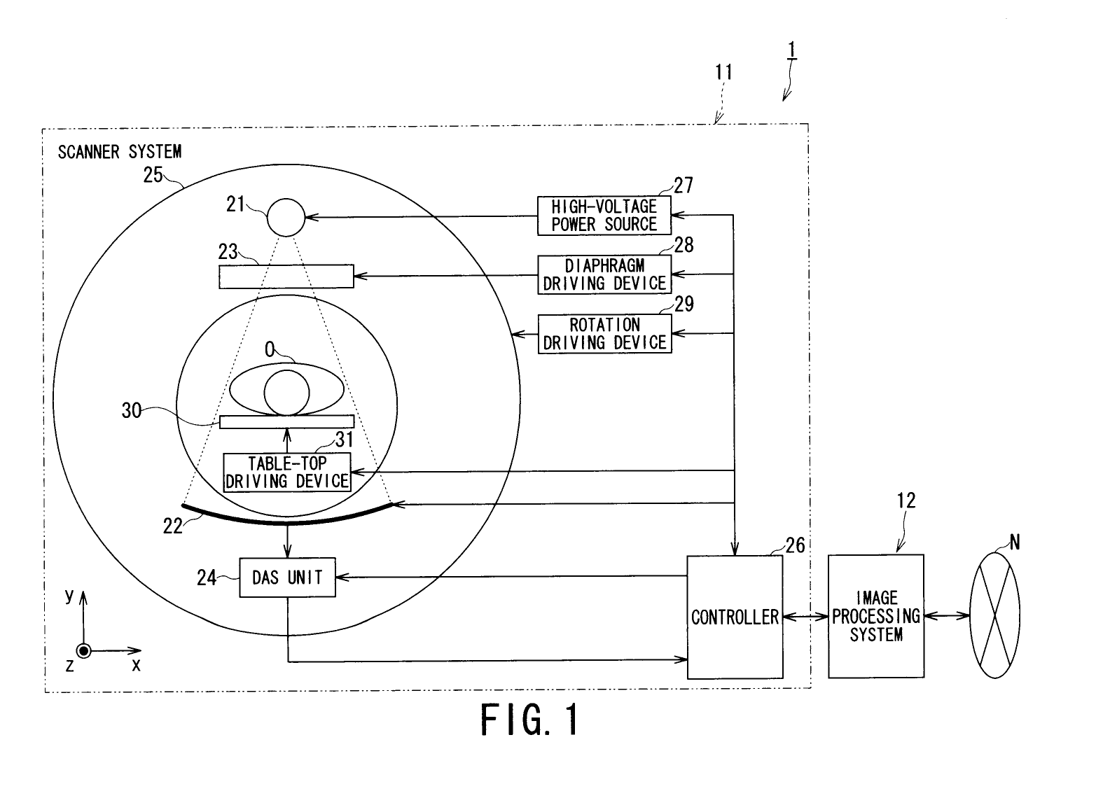

FIG. 1 is a hardware configuration diagram illustrating an X-ray CT apparatus according to the present embodiment.

FIG. 1 shows an X-ray CT apparatus 1 according to the present embodiment. The X-ray CT apparatus 1 mainly has a scanner system 11 and an image processing system 12. The scanner system 11 of the X-ray CT apparatus 1 is normally installed in an examination room, and generates X-ray transmission data on a shot area of an object (a human body) O. The image processing system 12 is normally installed in a control room next to the examination room, and generates projection data based on the transmission data to generate and display a reconstructed image.

The scanner system 11 of the X-ray CT apparatus 1 has an X-ray tube 21 as an X-ray source, an X-ray detector (a scintillation detector) 22, a diaphragm 23, a DAS (data acquisition system) unit 24, a rotation unit 25, a controller 26, a high-voltage power source 27, a diaphragm driving device 28, a rotation driving device 29, a table-top 30, and a table-top driving device (a table device) 31.

The X-ray tube 21 emits X-rays toward the X-ray detector 22 based on a tube voltage supplied from the high-voltage power source 27. The X-rays emitted from the X-ray tube 21 form a fan X-ray beam or a cone X-ray beam.

The X-ray detector 22 is a two-dimensional array-type X-ray detector 22 (also referred to as a multi-slice detector), which includes detection elements arranged in a matrix, that is, a plurality of (N) channels of detection elements in the channel direction perpendicular to the longitudinal direction of the table-top as the body axis direction, and a plurality of (M) rows of detection elements in the row direction. The X-ray detector 22 detects the X-rays emitted from the X-ray tube 21 and transmitted through the object O.

The diaphragm 23 regulates an emission range in the slice direction of the X-rays emitted from the X-ray tube 21 by the diaphragm driving device 28. To be more specific, the diaphragm driving device 28 regulates an opening of the diaphragm 23, so that the X-ray emission range in the slice direction can be changed.

The DAS unit 24 converts an electric signal of the transmission data detected by each detection element of the X-ray detector 22 into a voltage signal, amplifies the voltage signal, and converts the amplified signal into a digital signal. Output data from the DAS unit 24 is supplied to the image processing system 12 via the controller 26.

The rotation unit 25 is accommodated in a gantry (not shown) of the scanner system 11. The rotation unit 25 integrally holds the X-ray tube 21, the X-ray detector 22, the diaphragm 23, and the DAS unit 24. The rotation unit 25 can integrally rotate the X-ray tube 21, the X-ray detector 22, the diaphragm 23, and the DAS unit 24 around the object O with the X-ray tube 21 and the X-ray detector 22 facing each other.

The controller 26 includes a CPU (central processing unit), and a memory. The controller 26 conducts scanning by controlling the X-ray detector 22, the DAS unit 24, the high-voltage power source 27, the diaphragm driving device 28, the rotation driving device 29, and the table-top driving device 31 based on a control signal. The control signal is input from the image processing system 12.

The high-voltage power source 27 is controlled by the controller 26 to supply necessary power for emitting X-rays to the X-ray tube 21.

The diaphragm driving device 28 is controlled by the controller 26 to regulate the X-ray emission range in the slice direction of the diaphragm 23.

The rotation driving device 29 is controlled by the controller 26 to rotate the rotation unit 25 such that the rotation unit 25 is rotated around a hollow space while maintaining a positional relationship.

The table-top 30 can place the object O thereon.

The table-top driving device 31 is controlled by the controller 26 to move the table-top 30 along a z-axis direction. The rotation unit 25 has an opening in its center portion. The object O placed on the table-top 30 is inserted into the opening portion.

The image processing system 12 of the X-ray CT apparatus 1 is a computer-based device, and can communicate with a network N such as a hospital backbone LAN (local area network). Although not shown in the drawings, the image processing system 12 includes basic hardware such as a CPU, a memory, an HDD (hard disk drive), an input device, and a display device.

The image processing system 12 generates the projection data by performing correction processing (pre-processing) such as logarithmic conversion and sensitivity correction on raw data. The raw data is input from the DAS unit 24 of the scanner system 11. The image processing system 12 also eliminates scattered radiation from the projection data on which the pre-processing has been performed. The image processing system 12 eliminates scattered radiation based on a value of the projection data within the X-ray emission range. The image processing system 12 performs scattered radiation correction by reducing scattered radiation from target projection data to be subjected to the scattered radiation correction. The scattered radiation to be reduced is estimated from the magnitude of the value of the target projection data, or of projection data adjacent thereto. The image processing system 12 generates a reconstructed image based on the corrected projection data.

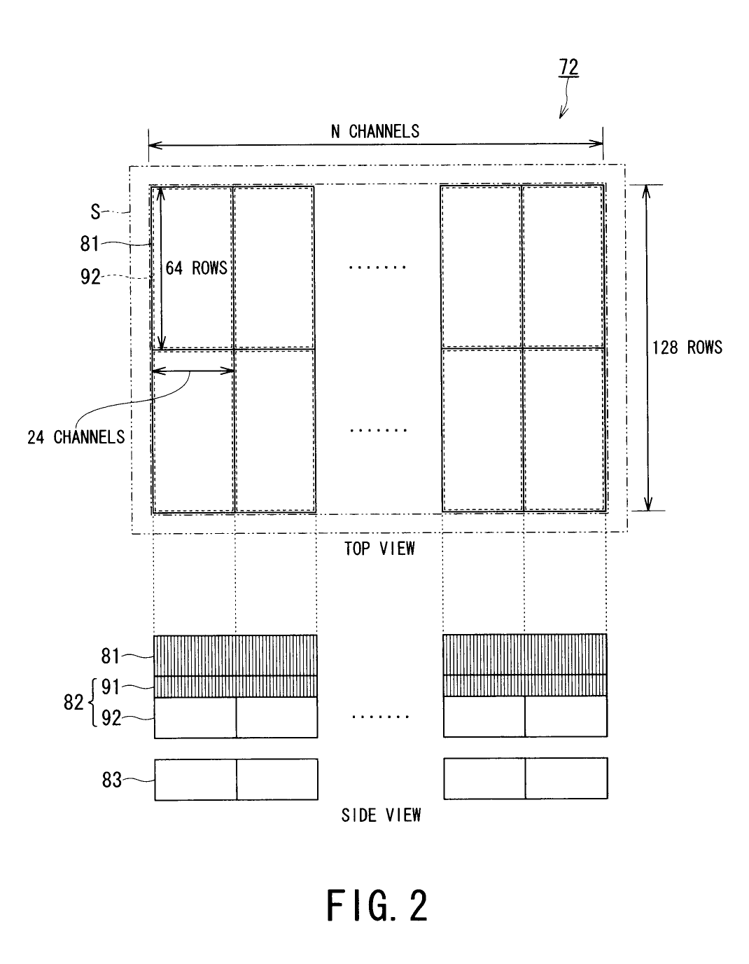

FIG. 2 is a top view (an X-ray incident surface) and a side view illustrating a first configuration of an X-ray detector of a conventional X-ray CT apparatus.

FIG. 2 shows an X-ray detector 72 of the conventional X-ray CT apparatus corresponding to N channels and 128 (M=128) rows. The X-ray detector 72 shown in FIG. 2 includes a plurality of collimator units 81 and detection element packs 82 arranged in the channel direction, and two collimator units 81 and detection element packs 82 arranged in the row direction. Each of the collimator units 81 is a group of collimators corresponding to 24 channels and 64 rows. Each of the detection element packs 82 includes a scintillator unit 91 and a PDA 92. The scintillator unit 91 is a group of scintillators respectively corresponding to the group of collimators, and the PDA 92 is a group of PDs respectively corresponding to the group of scintillators. The X-ray detector 72 also includes a plurality of DASs 83 arranged in the channel direction and two DASs 83 arranged in the row direction.

All the PDAs 92 (the detection element packs 82) shown in FIG. 2 are partially not adjacent to another PDA 92 but in contact with a working area S. Thus, in a process of arraying the PDAs 92 in the X-ray CT apparatus, all the PDAs 92 can be arrayed using the working area S. However, since there is a minimum gap necessary for arraying the PDAs 92 between respective adjacent PDAs 92, the PDAs 92 are difficult to accurately array in a two-dimensional direction in the process of arraying the PDAs 92 in the X-ray CT apparatus.

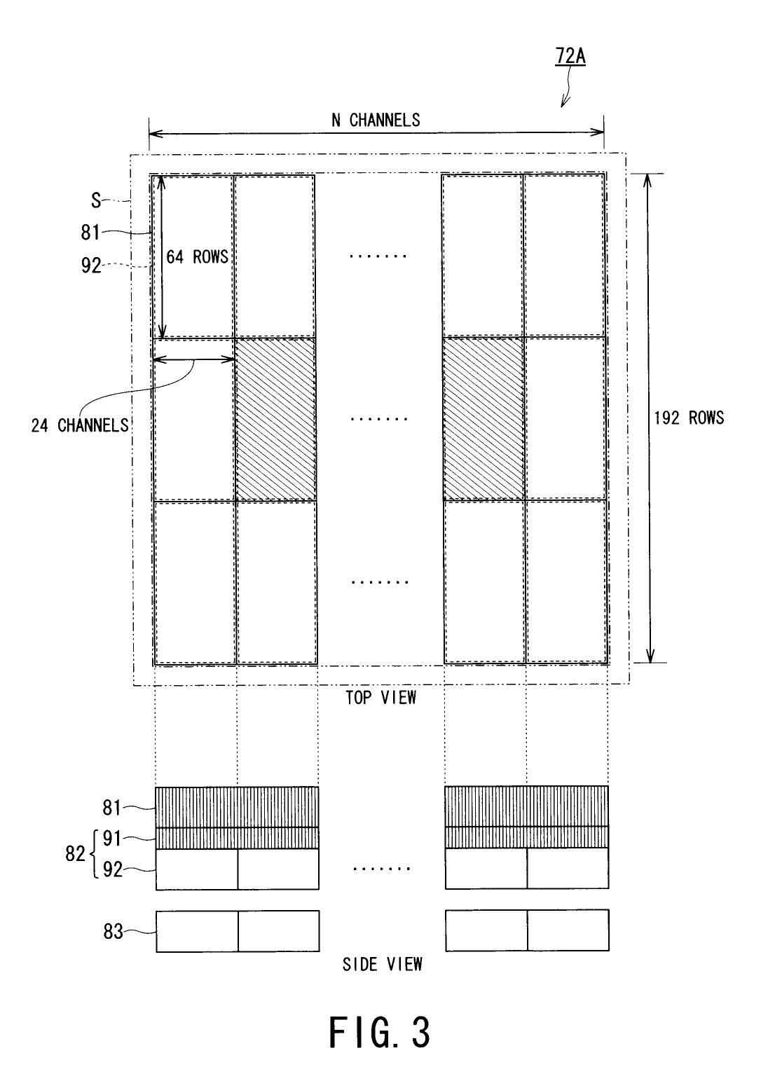

FIG. 3 is a top view (an X-ray incident surface) and a side view illustrating a second configuration of the X-ray detector of the conventional X-ray CT apparatus.

FIG. 3 shows an X-ray detector 72A of the conventional X-ray CT apparatus corresponding to N channels and 192 (M=192) rows. The X-ray detector 72A shown in FIG. 3 includes the plurality of collimator units 81 and detection element packs 82 arranged in the channel direction, and three collimator units 81 and detection element packs 82 arranged in the row direction. Each of the detection element packs 82 included the scintillator unit 91 and the PDA 92. The X-ray detector 72A also includes the plurality of DASs 83 arranged in the channel direction and three DASs 83 arranged in the row direction.

Since some of the PDAs 92 (the detection element packs 82) shown in FIG. 3 are partially not adjacent to another PDA 92 but in contact with the working area S. Thus, in the process of arraying the PDAs 92 in the X-ray CT apparatus, some of the PDAs 92 can be arrayed using the working area S. However, since there is a minimum gap necessary for arraying the PDAs 92 between respective adjacent PDAs 92, the PDAs 92 are difficult to accurately array in the two-dimensional direction in the process of arraying the PDAs 92 in the X-ray CT apparatus.

There are also PDAs 92 (a shaded area in FIG. 3) not in contact with the working area S in the X-ray detector 72A where the three or more PDAs 92 are arranged in the row direction. The PDA 92 not in contact with the working area S is entirely surrounded by other PDAs 92. In the process of arraying the PDAs 92 in the X-ray CT apparatus, the PDAs 92 not in contact with the working area S are difficult to array when there is only a minimum gap necessary for arraying the PDAs 92 in contact with the working area S. To solve the problem, the gap between the PDAs 92 is made larger than the minimum gap necessary for arraying the PDAs 92 in contact with the working area S, so that the PDAs 92 not in contact with the working area S can be accurately arrayed. When the gap between the PDAs 92 is made larger than the minimum gap necessary for arraying the PDAs 92 in contact with the working area S, however, detection efficiency of X-rays is reduced.

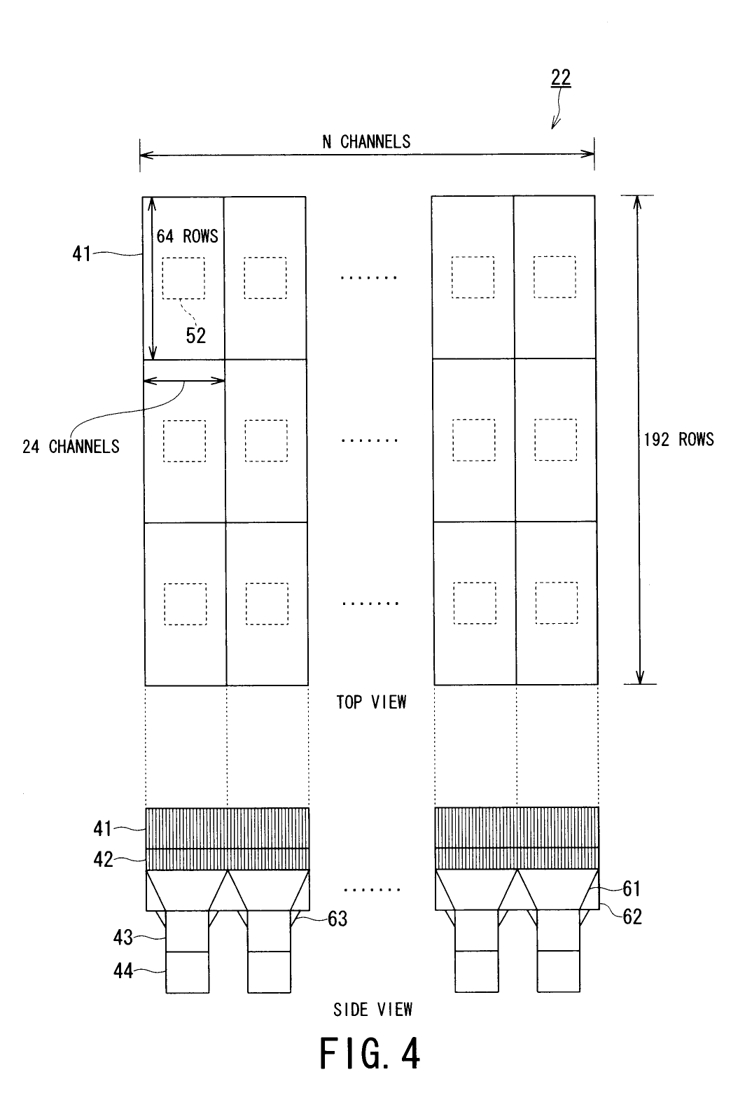

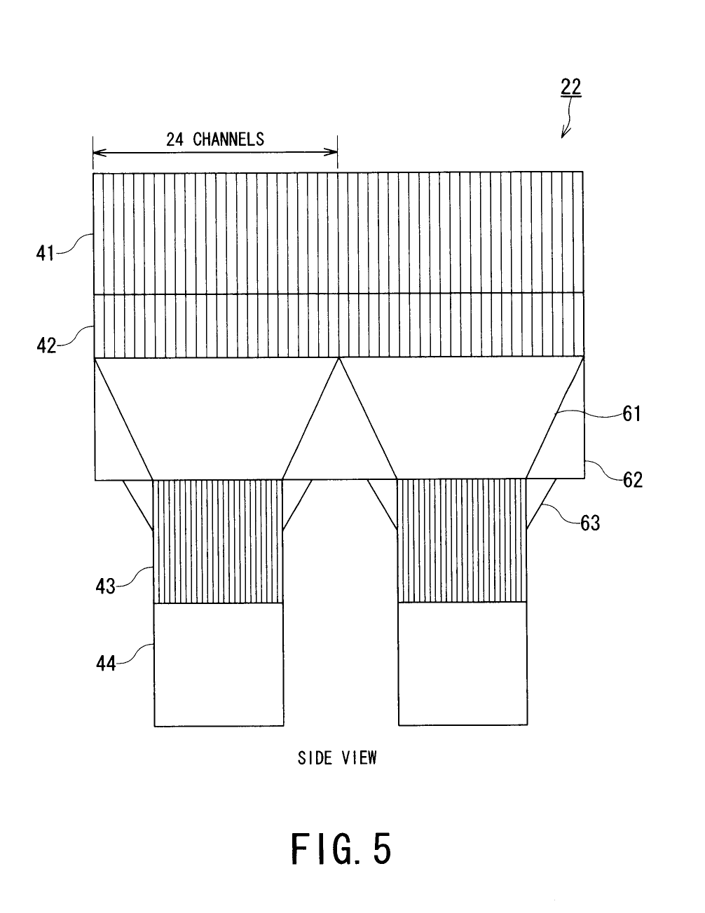

FIG. 4 is a top view (an X-ray incident surface) and a side view illustrating a configuration of the X-ray detector 22 of the X-ray CT apparatus 1 according to the present embodiment. FIG. 5 is an enlarged side view illustrating a first configuration example of the X-ray detector 22 of the X-ray CT apparatus 1 according to the present embodiment.

FIG. 4 shows the X-ray detector 22 of the X-ray CT apparatus 1 according to the present embodiment corresponding to N channels and 192 (M=192) rows. The X-ray detector 22 shown in FIG. 4 includes a plurality of collimator units 41, scintillator units 42 and PDAs 43 arranged in the channel direction, and three collimator units 41, scintillator units 42 and PDAs 43 arranged in the row direction. Each of the collimator units 41 is a group of collimators corresponding to 24 channels and 64 rows. Each of the scintillator units 42 is a group of scintillators respectively corresponding to the group of collimators. Each of the PDAs 43 is a group of PDs respectively corresponding to the group of scintillators. The X-ray detector 22 also includes a plurality of DASs 44 arranged in the channel direction, and three DASs 44 arranged in the row direction. The DASs 44 constitute the DAS unit 24.

Although not shown in the drawings, a partition wall is provided between respective collimators of the collimator unit 41. Although not shown in the drawings, the DAS 44 includes a plurality of DAS chips (C-amp chips and A/D conversion chips) corresponding to the number of the PDs of the PDA 43 on a one-to-one basis, or a plurality of DAS chips corresponding to the number of the PDs on a one-to-plural basis.

The X-ray detector 22 includes a light guide 61, an engagement frame 62 configured to hold the light guide 61, and an engagement stay 63 provided on a surface on the PDA 43 side of the engagement frame 62. The light guide 61 works as a convex lens. The light guide 61 has such a shape as to guide fluorescence to each PD of the PDA 43 while focusing the fluorescence toward the PDA 43. The fluorescence is emitted from each scintillator of the scintillator unit 42. A space for fixing the engagement stay 63 can be thereby provided on the surface on the PDA 43 side of the light guide 61. The engagement frame 62 has a grid shape when viewed from a lower surface (a surface facing the X-ray incident surface). The engagement frame 62 may be made of metal or resin, for example.

The X-ray detector 22 has a structure in which the scintillator unit 42 and the PDA 43 are separated from each other, and the collimator unit 41, the scintillator unit 42, the light guide 61, the engagement frame 62, and the engagement stay 63 are integrated. The X-ray detector 22 also has a structure in which the PDA 43 is engageable with the integrated collimator unit 41, scintillator unit 42, light guide 61, engagement frame 62, and engagement stay 63 via the engagement stay 63. The light guide 61 and the PDA 43 are bonded together with an optical adhesive, so that the fluorescence can be guided from the light guide 61 to the PDA 43 with a small loss.

A function of the X-ray detector 22 will be described based on FIG. 5. Each collimator of the collimator unit 41 collimates X-rays transmitted through the object O. Each scintillator of the scintillator unit 42 emits fluorescence based on the X-rays collimated by each collimator of the collimator unit 41. The light guide 61 focuses the fluorescence emitted from each scintillator of the scintillator unit 42. Each PD of the PDA 43 converts the focused fluorescence into an electric signal. The electric signal from the PDA 43 is output to the DAS 44.

In the X-ray detector 22 shown in FIG. 4, even when three or more PDAs 43 are arranged in the row direction, there is a gap large enough to array the PDAs 43 between respective adjacent PDAs 43 in a process of arraying the PDAs 43 in the X-ray CT apparatus 1. The PDAs 43 are also easily attached and detached. The PDAs 43 can be thereby accurately arrayed in the two-dimensional direction.

Moreover, even when three or more PDAs 43 are arranged in the row direction, the fluorescence from the scintillator unit 42 is focused onto the small-sized PDA 43 without changing a gap between the scintillator units 42. A decrease in detection efficiency of X-rays can be thereby prevented.

The X-ray detector 22 is not limited to the structure shown in FIG. 4 in which the collimator unit 41, the scintillator unit 42, the light guide 61, the engagement frame 62, and the engagement stay 63 are integrated. For example, in the X-ray detector 22, the collimator unit 41 and the scintillator unit 42 may be integrated. The light guide 61 may be mounted on the PDA 43 via the engagement stay 63 fixed to the engagement frame 62, and the PDA 43 may be freely attached to and detached from the integrated collimator unit 41 and scintillator unit 42. As a third configuration, the collimator unit 41, the scintillator unit 42, the PDA 43, and the light guide 61 may be integrated.

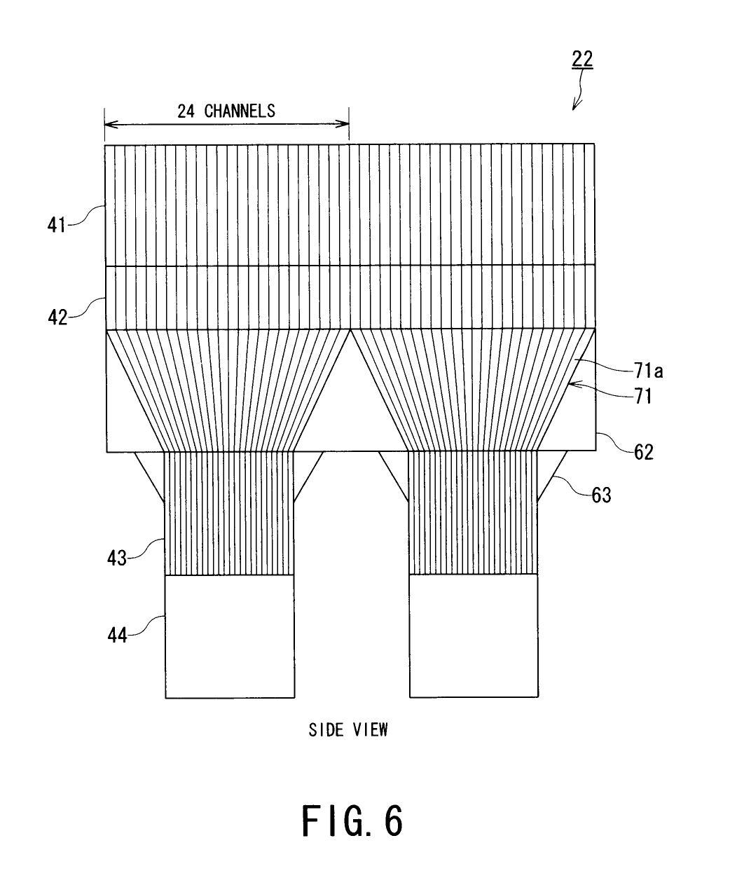

FIG. 6 is an enlarged side view illustrating a second configuration example of the X-ray detector 22 of the X-ray CT apparatus 1 according to the present embodiment.

In the second configuration example of the X-ray detector 22 shown in FIG. 6, the X-ray detector 22 includes a light guide 71. The light guide 71 includes light guide elements 71a as a group of light guide elements respectively corresponding to the group of scintillators. The light guide 71a guides the fluorescence emitted from each scintillator of the scintillator unit 42 to each PD of the PDA 43.

A function of the X-ray detector 22 will be described based on FIG. 6. Each collimator of the collimator unit 41 collimates X-rays transmitted through the object O. Each scintillator of the scintillator unit 42 emits fluorescence based on the X-rays collimated by each collimator of the collimator unit 41. Each light guide element 71a guides the fluorescence emitted from each scintillator of the scintillator unit 42. Each PD of the PDA 43 converts the fluorescence into an electric signal. The electric signal from the PDA 43 is output to the DAS 44.

In the X-ray CT apparatus 1 according to the present embodiment, the PDA 43 is reduced in size, and the fluorescence emitted from the scintillator unit 42 is effectively guided to the PDA 43. The PDAs 43 can be easily accurately arrayed without reducing the detection efficiency of X-rays. In addition, the number of channels and rows of the PDA stated above are only one case, and the present inventions are not limited to this.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An X-ray CT apparatus comprising:
an X-ray source configured to generate an X-ray; and
an X-ray detector configured to acquire an electric signal based on the X-ray, wherein
the X-ray detector comprising:
a plurality of collimators configured to collimate the X-ray;
a plurality of scintillators respectively configured to emit fluorescence based on the X-ray from the plurality of collimators;
a plurality of photodiodes respectively configured to convert the fluorescence from the plurality of scintillators into the electric signal; and
a light guide having such a shape as to guide the fluorescence emitted from the plurality of scintillators respectively to the plurality of photodiodes while focusing the fluorescence toward the plurality of photodiodes.

2. The X-ray CT apparatus according to claim 1, wherein
the X-ray detector includes three or more sets of the plurality of photodiodes in a row direction.

3. The X-ray CT apparatus according to claim 1, wherein
the plurality of photodiodes are fixed to the light guide side by using a space in a width direction of the plurality of photodiodes obtained by reducing a width of the plurality of photodiodes to be smaller than that of the plurality of scintillators.

4. The X-ray CT apparatus according to claim 3, further comprising:
a frame configured to hold the light guide; and
a stay provided on a surface of the frame on a side of the plurality of photodiodes to be engageable with the plurality of photodiodes.

5. The X-ray CT apparatus according to claim 4, wherein
the plurality of collimators, the plurality of scintillators, the light guide, the frame and the stay are integrated.

6. An X-ray detector comprising:
a plurality of collimators configured to collimate an X-ray;
a plurality of scintillators respectively configured to emit fluorescence based on the X-ray from the plurality of collimators;
a plurality of photodiodes respectively configured to convert the fluorescence from the plurality of scintillators into the electric signal; and
a light guide having such a shape as to guide the fluorescence emitted from the plurality of scintillators respectively to the plurality of photodiodes while focusing the fluorescence toward the plurality of photodiodes.

7. The X-ray detector according to claim 6, wherein
the X-ray detector includes three or more sets of the plurality of photodiodes in a row direction.

8. The X-ray detector according to claim 6, wherein
the plurality of photodiodes are fixed to the light guide side by using a space in a width direction of the plurality of photodiodes obtained by reducing a width of the plurality of photodiodes to be smaller than that of the plurality of scintillators.

9. The X-ray detector according to claim 8, further comprising:
a frame configured to hold the light guide; and
a stay provided on a surface of the frame on a side of the plurality of photodiodes to be engageable with the plurality of photodiodes.

10. The X-ray detector according to claim 9, wherein
the plurality of collimators, the plurality of scintillators, the light guide, the frame and the stay are integrated.
